(19) United States
(12) Patent Application Publication
(10) Pub. No.: US 2002/0156501 A1
(43) Pub. Date: Oct. 24, 2002

(54) Method of relieving dizziness or vertigo

(76) Inventors: Thomas L. Grey, Carlsbad, CA (US); Gregory j. Gruzdowich, Carlsbad, CA (US)

Correspondence Address: Crockett & Crockett, Suite 400, 24012 Calle De La Plata, Laguna Hills, CA, 92653, US

(21) Appl. No.: 09/838,537

(22) Filed: Apr. 18, 2001

(51) Int. Cl. A61N 1/36

(52) U.S. Cl. 607/2

(57) ABSTRACT

A device for providing noninvasive electrical stimulation of a single acupuncture site for treatment of dizziness, vertigo, and/or the symptoms associated with Meniere's disease is disclosed.

### FIELD OF THE INVENTION

The methods and devices described below relate to the fields of treatment of dizziness, vertigo, and/or the symptom's associated with Meniere's disease and noninvasive electrical stimulation of an acupuncture point.

### BACKGROUND OF THE INVENTION

Dizziness is a feeling of faintness or an inability to keep normal balance in a standing or sitting position. The causes of dizziness are many. Vertigo is a type of dizziness characterized by a sensation of rotation or movement of one's self (subjective vertigo) or of one's surroundings (objective vertigo). Vertigo results mainly from diseases of the inner ear. Treatments for vertigo include medications such as antihistamines, anticholinergics, and sedative-hypnotics. These medications all have side-effects which the patient may wish to avoid.

Meniere's disease is an abnormality of the inner ear that causes a host of symptoms including severe dizziness and/or vertigo. Currently there is no cure for Meniere's disease. Operations may reverse the disease process, but the effectiveness of these operations has been difficult to establish. As with any inner ear operations, there is a risk of hearing loss. The most common surgery performed entails inserting a shunt into the inner ear to drain off excess fluid. A more reliable surgery called a vestibular neurectomy, includes severing the vestibular nerve. The vestibular nerve aids in balance and severing the nerve prevents it from sending distorted messages to the brain. A problem with this surgery is that the vestibular nerve is very close to hearing and facial nerves. Thus, the risk of affecting a patient's hearing and/or facial muscle control is increased with this surgery. Further, older patients often have difficultly recovering from this surgery.

A radical surgery used to treat Meniere's disease is a labyrinthectomy which includes removal of the membranous labyrinth. This is an irreversible procedure that is often successful in eliminating the dizziness associated with Meniere's disease. Unfortunately this procedure results in a total loss of hearing. Further, labyrinthectomies themselves may result in other balance problems. All of these surgeries have very severe potential side-effects, and thus additional treatment options are always being sought.

Some other treatments currently being used which do not have such severe side-effects include a change of diet. Eliminating caffeine, alcohol, and salt may relieve the frequency and intensity of the attacks. Also eliminating tobacco use and relieving stress may lessen the severity of the symptoms. Additionally, medications that control allergies, reduce fluid retention or improve blood circulation in the inner ear may help.

Acupuncture has long been used in the treatment of Meniere's disease and various types of dizziness. In accordance with well-known acupuncture standards, several acupuncture points are simultaneously stimulated to achieve the therapeutic goal. As taught in The Basics of Acupuncture by Stux and Pomeranz, Springer-verlag, New York, pp. 237-238, 1995, ten acupuncture points are stimulated: the top of the head (Du 20 Baihui), in front of the ear (SJ.21 Ermen), front of ear (SI.19 Tinggong), in front of the ear (GB.2 Tinghui), topside of hand (SJ.3 Zhongzhu), topside of lower arm (SJ.5 Waiguan), (LI.4 Hegu), side of lower arm (SI.6 Yanglao), top of the toe (GB.41 Foot Linqi), and the foot (Liv.3 Taichong).

Bertolucci, Nausea Control Device, U.S. Pat. No. 4,981,146, Jan. 1, 1991, describes a nausea control device in the form of a watch-like housing attachable to the human wrist by an adjustable attachment band. The device uses non-invasive nerve stimulation whereby electricity is passed through two electrodes to stimulate nerves located on the ventral side of the wrist (this anatomical position is sometimes referred to as the palmar side of the wrist). The treatment provided by the device is sometimes referred to as electro-acupuncture which is a form of acupuncture, and the ventral site of application is referred to in the acupuncture art as the P6 point, pericardium 6 point, or master point of the pericardium meridian (sometimes referred to as the vascular meridian). A primary object of the invention is to provide a non-chemical, non-invasive, painless and inexpensive method of alleviating nausea. It is also portable, self-contained and convenient to the patient. Electrical pulse repetition rate of approximately 70 pulses per second and a pulse width of 80 microseconds has been found to provide effective relief of nausea in a patient. Our currently preferred electrical pulse pattern comprises about 350 microsecond pulse width at about 31 pulses per second at power levels of about 10-35 milli-amps peak pulse height. Thus a wide range of pulse patterns may be used in non-invasive nerve stimulation devices.

We have discovered that using noninvasive electrical stimulation of the P6 or Neiguan point of the pericardium meridian relieves dizziness, vertigo, and/or the symptoms associated with Meniere's disease.

### SUMMARY OF THE INVENTIONS

The method described below employs use of the device described in Bertolucci, Nausea Control Device, U.S. Pat. No. 4,981,146 (Jan. 1, 1991), and similar devices, for the relief and alleviation of dizziness, vertigo, and/or the symptoms associated with Meniere's disease.

### BRIEF DESCRIPTION OF THE DRAWINGS

FIG. 1 illustrates placement of an electro-acupuncture device over the P6 acupuncture point on the human wrist.

FIG. 2 illustrates a stimulation waveform for stimulating the wrist in accomplishing the treatment.

FIG. 3 illustrates an individual pulse of the stimulation waveform.

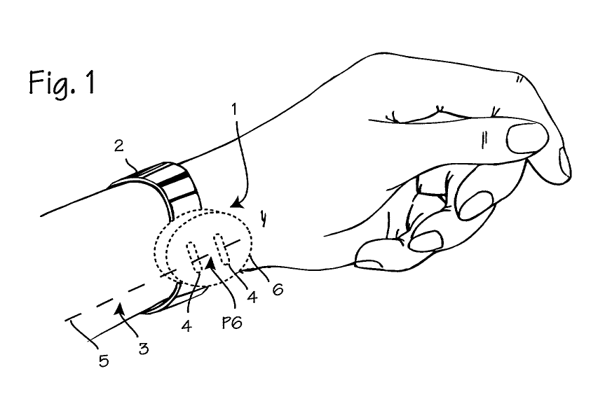

### DETAILED DESCRIPTION OF THE PREFERRED EMBODIMENT

Use of our ReliefBand® NST™ device for the approved treatment of nausea has revealed that the treatment also relieves dizziness and/or the symptoms associated with Meniere's disease. Significant reduction in dizziness, vertigo, and Meniere's symptoms have been observed when electrostimulation is provided to the P6 point on the wrist. The ReliefBand® NST™ is a wristwatch like device worn on the wrist and energized to provide electrical stimulation to the wrists. The ReliefBand® NST™ non-invasive nerve stimulation device 1 is secured with strap 2 to the ventral side of the wrist 3 such that the pair of electrodes 4 are disposed over the median nerve 5 (indicated by the phantom line) in contact with the skin in the vicinity of the P6 acupuncture point. The electrodes are on the underside of the housing 6, the required battery and control electronics are housed within the housing, and input mechanisms are located on the outer face of the housing. The electrodes stimulate the median nerve and collateral or associated nerve structures.

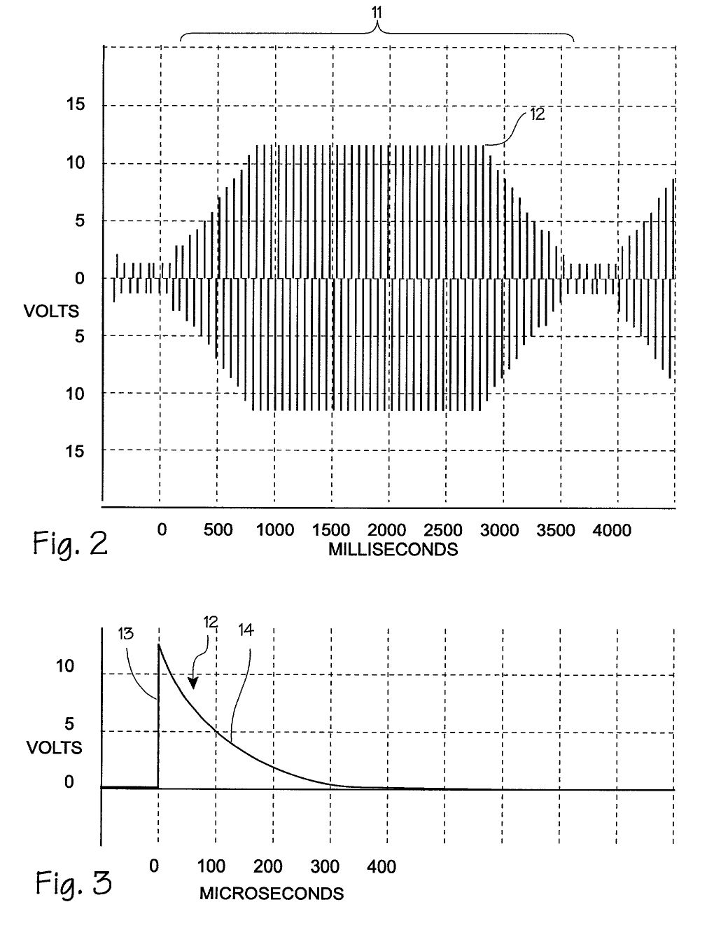

FIG. 2 shows the preferred waveform. The overall waveform comprises a series of bipolar trapezoidal waveforms which make low frequency pulses 11. The waveform is initiated at low power levels of about 1 to 2 volts and ramps up over a period of about 1 second to a maximum level of 10-20 volts, and is maintained for about 2 seconds, and then ramps down over a period of about 1 second to low power levels of about 1 to 2 volts. The individual pulses 12 are separated by about 32 milliseconds (msec)(measured peak to peak), and last about 350 microseconds (μsec). The individual pulses alternate between negative and positive pulses, and are said to constitute a bipolar waveform. The individual pulses are illustrated in FIG. 3, in which the time scale is enlarged to show the detail. The individual pulse 12 is made of a sharply vertical spike which decays exponentially over a period of about 350 μsec, thus comprising a basically vertical leading edge 13 and an exponentially decaying trailing edge 14 to each individual pulse. The following pulse will be shaped the same, except that it will be of negative voltage. The exponential nature of the individual pulse decay maximizes the high frequency components in the signal. These high frequency components contribute to a lessening of the skin impedance, in particular the capacitive components. This contributes to a higher level of current able to enter the deeper tissues. The power levels may be adjusted up or down to intensify the therapeutic effect of the device or lessen the sensation causes by the device, according to the preferences of individual users. The pulse rate within the waveform may be increased or decreased also.

To use the device to alleviate dizziness and/or Meniere's symptoms, the user merely secures the housing over the inner surface of the wrist and straps it on like a wristwatch. This places the electrodes over the P6 acupuncture point, in electrical contact with the skin overlying the median nerve. The user then turns the device on, adjusts it to a comfortable power level, and allows stimulation to continue for a few minutes, for example 5-10 minutes to achieve relief. The device may be applied intermittently, once every hour or so, or continuously. The device provides electrical current and voltage to the electrodes which stimulates the P6 acupuncture point. While less convenient, the methods may be accomplished with electro-acupuncture needles or electrodes handled individually by an acupuncturist.

While the devices and methods have been described in reference to the environment in which they were developed, they are merely illustrative of the principles of the inventions. Other embodiments and configurations may be devised without departing from the spirit of the inventions and the scope of the appended claims.

We claim:

1. A method of relieving dizziness in a patient comprising the steps of:
mounting a non-invasive nerve stimulation device onto the ventral side of the wrist;
generating a stimulation signal; and
delivering the stimulation signal to the ventral side of the wrist.

2. A method of relieving dizziness in a patient comprising the steps of:
mounting at least one electrode onto the ventral side of the wrist;
generating a stimulation signal; and
delivering the stimulation signal to said at least one electrode to stimulate the ventral side of the wrist.

3. The method of claim 2 wherein said mounting step includes providing a wristwatch-like housing carrying the electrodes, and providing securing means for mounting the housing onto the wrist, with the housing having a circuit means for generating the stimulation signal encased within the housing.

4. The method of claim 2 wherein said delivering step comprises delivering an intermittent stimulation signal.

5. The method of claim 2 wherein said delivering step comprises delivering a continuous stimulation signal.

6. A method of relieving dizziness in a patient by stimulating the median nerve and associated nerve structures in the wrist of the patient with electrical energy.

7. A method of relieving dizziness in a patient comprising the steps of:
applying an electro-acupuncture device to the wrist; and
stimulating the wrist with the electro-acupuncture device.

8. A method of relieving vertigo in a patient comprising the steps of:
mounting a non-invasive nerve stimulation device onto the ventral side of the wrist;
generating a stimulation signal; and
delivering the stimulation signal to the ventral side of the wrist.

9. A method of relieving vertigo in a patient comprising the steps of:
mounting at least one electrode onto the ventral side of the wrist;
generating a stimulation signal; and
delivering the stimulation signal to said at least one electrode to stimulate the ventral side of the wrist.

10. The method of claim 9 wherein said mounting step includes providing a wristwatch-like housing carrying the electrodes, and providing securing means for mounting the housing onto the wrist, with the housing having a circuit means for generating the stimulation signal encased within the housing.

11. The method of claim 9 wherein said delivering step comprises delivering an intermittent stimulation signal.

12. The method of claim 9 wherein said delivering step comprises delivering a continuous stimulation signal.

13. A method of relieving vertigo in a patient by stimulating the median nerve and associated nerve structures in the wrist of the patient with electrical energy.

14. A method of relieving vertigo in a patient comprising the steps of:
applying an electro-acupuncture device to the wrist; and
stimulating the wrist with the electro-acupuncture device.
